(19) United States
(12) Patent Application Publication
(10) Pub. No.: US 2005/0059948 A1
(43) Pub. Date: Mar. 17, 2005

(54) Attachable side panels for absorbent garments

(76) Inventor: Kathy Murguly, Hillsborough, NJ (US)

Correspondence Address: ROPES & GRAY LLP, ONE INTERNATIONAL PLACE, BOSTON, MA, 02110-2624, US

(21) Appl. No.: 10/942,244

(22) Filed: Sep. 16, 2004

Related U.S. Application Data
(60) Provisional application No. 60/503,532, filed on Sep. 16, 2003.

(51) Int. Cl. A61F 13/15

(52) U.S. Cl. 604/387

(57) ABSTRACT

The present invention discloses a single attachable side panel for use with an absorbent article. The side panel comprises a sheet of skin-friendly material, having a first edge and a second edge extending in a lengthwise direction, and a third edge and a fourth edge extending in a widthwise direction. The side panel also includes a fastener, positioned along the third and/or fourth edge and middle portion, adapted to affix said sheet to an absorbent article. The absorbent article can be a conventional diaper or other incontinent product, which is positioned between the legs of the wearer and having a portion positioned in the front and back of the wearer. The side panel can then allow for the attachment of the front portion to the back portion of the absorbent article securing the absorbent article about the wearer.

### CROSS-REFERENCE TO RELATED APPLICATIONS

This patent application claims the benefit of, and incorporates by reference in its entirety, prior provisional patent application U.S. Provisional Patent Application No. 60/503,532, filed Sep. 16, 2003.

### BACKGROUND OF THE INVENTION

The marketplace provides various size adult absorbent garments (small, medium, large and extra large) for incontinent patients. The extra large size has the largest waist circumference of up to a maximum of 63 to 65 inches. Unfortunately, there are some incontinent patients who require garments that can provide for a waist circumference greater than 65 inches. For these individuals, there are no commercially available products that meet their needs.

Manufacturing adult absorbent garments with waist circumferences greater than 65 inches would require specialized, expensive production equipment, capable of handling extremely wide and heavy material rolls. They would also require wider adhesive application systems, wider rotary cutting knives, larger packaging equipment, among other things. When taking all of this into consideration, along with the fact that there are not many incontinent users of this large sized product, one can see why there are no manufacturers of this type of product today. A commercial need exists to provide a product that is a cost effective alternative.

### SUMMARY OF THE INVENTION

The present invention discloses an attachable side panel for use with an absorbent article. The side panel comprises a sheet of skin-friendly material, having a first edge and a second edge extending in a lengthwise direction, and a third edge and a fourth edge extending in a widthwise direction. The side panel also includes a fastener, positioned along the third and/or fourth edge and middle portion adapted to affix said sheet to an absorbent article. The absorbent article can be a conventional diaper or other incontinent product, which is positioned between the legs of the wearer, with a portion of the article positioned in the front and a portion in the back of the wearer. The side panel allows for the attachment of the front portion to the back portion of the absorbent article securing the absorbent article about the wearer.

In an alternative embodiment, the present invention discloses a two-piece absorbent article comprising a side panel and an absorbent article. The side panel has a sheet of skin-friendly material with a first and a second edge extending in a lengthwise direction, and a third and a fourth edge extending in a widthwise direction. The side panel includes a fastener, positioned along the third and fourth edges and middle portion, adapted to affix said sheet to an absorbent article; and an absorbent article, having a center portion with an absorbent core that is positioned between the legs of a wearer, a front portion that is positioned in front of the wearer; and a back portion positioned in the back of the wearer, wherein the side panels attach to the front and back portion of said absorbent article securing it about both sides of the wearer.

### BRIEF DESCRIPTION OF THE DRAWINGS

FIG. 1A is a top view of one embodiment of the invention showing a side panel with fasteners positioned along one edge and in the middle portion of the side panel. One of these panels is used to secure an absorbent article about a wearer.

FIG. 1B is a top view of the same embodiment as in FIG. 1A with the attachment means positioned intermittently along one edge and in the middle portion of the side panel.

FIG. 1C is a top view of the same embodiment as in FIG. 1A with the fastener positioned along both edges and in the middle portion of the side panel.

FIG. 2 is a perspective view illustrating how the side panel works with a standard absorbent article.

FIG. 3A is a top view of another embodiment of the side panel of the present invention showing a central elastic section and two non-elastic sections. The side panel includes a release tape and positioning adhesive.

FIG. 3B is a cross sectional view showing how the side panel of the present invention can be constructed.

### DETAILED DESCRIPTION OF THE INVENTION

The present invention provides a means for incontinent bariatric users to be able to use commercially available adult brief products. This is achieved by providing side panels that attach to each side of any commercially available adult absorbent article, thereby allowing the product to go beyond its widest allowable circumference.

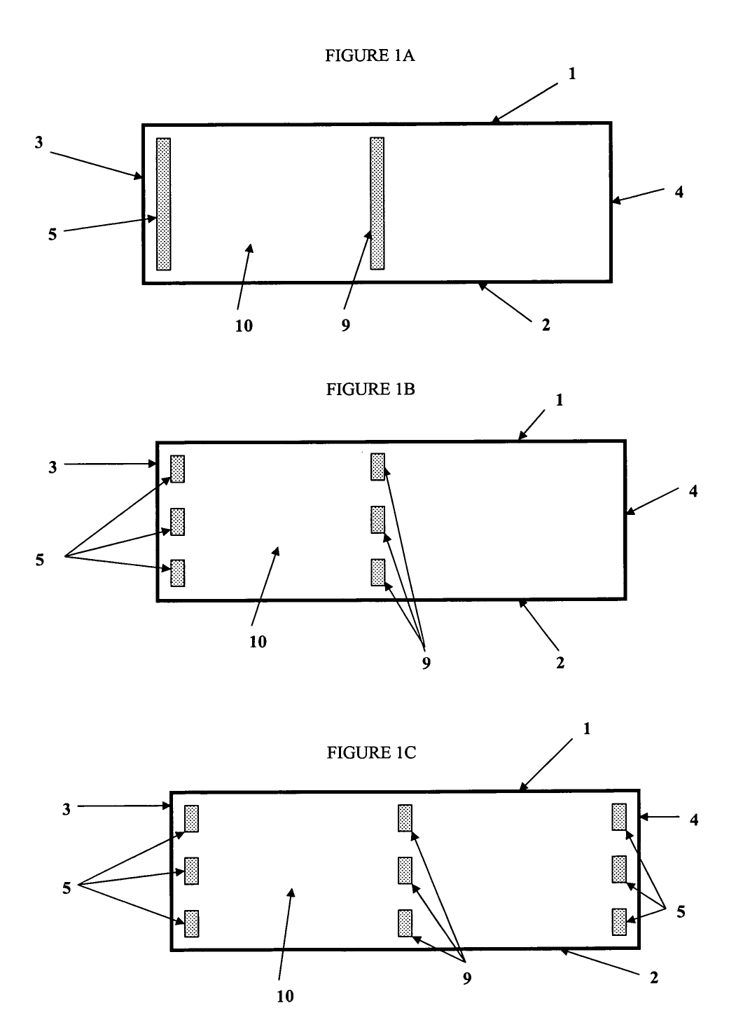

FIGS. 1A-1C illustrate the single attachable side panel of the present invention. In these Figures, the side panel comprises a sheet of skin-friendly material 10 having first edge 1 and second edge 2 extending in a lengthwise direction, and third edge 3 and fourth edge 4 extending in a widthwise direction. The side panel includes first fastener 5 positioned on third edge 3, and second fastener 9 positioned in the middle of the third and fourth edge as shown in FIG. 1A. In the alternative, the first fastener can be positioned on fourth edge 4. The fasteners can be applied in a single strip or intermittently (shown in FIG. 1B), or in any other manner as long it allows the side panel to sufficiently attach to the absorbent garment, and in particular its upper and lower corners. The fasteners can be positioned on both edges as shown in FIG. 1C along with the middle fastener.

In yet another embodiment, the third edge and the fourth edge can be wider than the width of the middle portion of the side panel. The side panel can also have a third edge and a fourth edge narrower than the middle portion, similar in shape to a cumber bund.

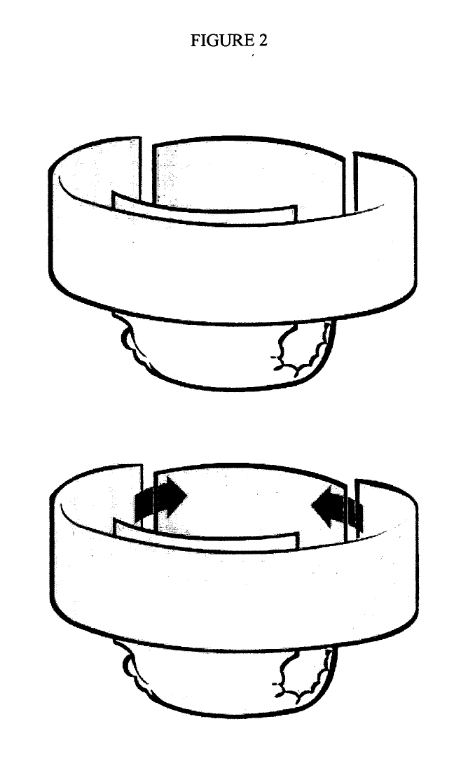

FIG. 2 illustrates how the fastener of one of the side panels of the invention is adapted to affix said sheet to an absorbent article positioned between the legs of a wearer having a portion positioned in the front and the back of the wearer, wherein said side panel attaches to the front and back portion of the absorbent article allowing the absorbent article to be secured about one side of the wearer. It is understood that the side panel of the present invention can cross over either the front or back portion of the diaper.

The sheet used to make the side panel of the present invention can be made of various skin-friendly materials. The term “skin-friendly”, as used herein, is intended to mean any comfortable material, including breathable material, that will mitigate any kind of discomfort to the wearer when in contact with their skin. One such material is poly laminates commercially available from Clopay Plastic Products of Mason, Ohio. Another acceptable material is a non-woven fabric, such as SBPP (Spundbond Polypropylene) or SMS (Spunbond-Meltblown-Spunbond). Such non-woven materials are commercially available from SAAF Advanced Fabrics of Al-Ahsa, Saudi Arabia. Yet other acceptable materials are other non-woven fabrics such as ABPET (Adhesive Bonded Polyester), TABICO (Through Air Bonded Bi-Component Fiber), TBPP (Thermal Bonded polypropylene), spunlace fibers, hydro-entangled fibers, needle-punched fibers, airlaid composites and woven fabrics. These materials are commercially available from PGI Nonwovens of Mooresville, N.C.

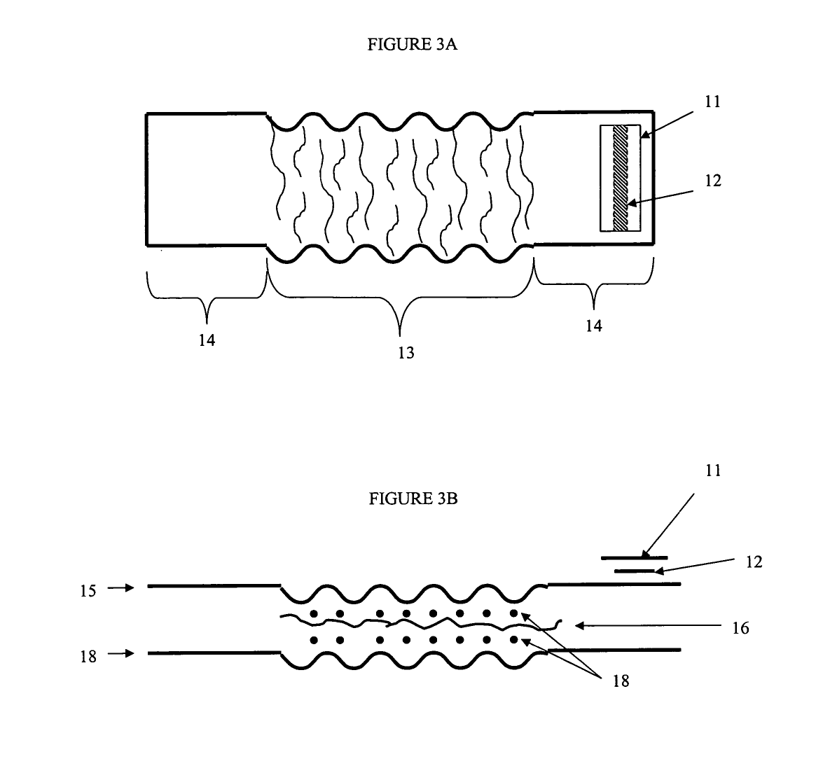

The sheet of the present invention can also be made of an elastic composite, comprising an elastic material sandwiched in-between two layers of non-woven fabric. The elastic composite can also be bonded to one or more non-stretchable materials in sections (not illustrated). Such elastic composites are commercially available from Clopay Plastic Products of Mason, Ohio. The sheet can have an elastic section and a non-elastic sections as needed to enable the panel to give a better fit. FIG. 3B, is a cross section of how the elastic sheet in FIG. 3A can be constructed with release paper 11, positioning paper 12, first sheet 15, one or more elastics 16, adhesive 17 and second sheet 18.

In addition, the sheet of the present invention can also comprise sheets of breathable poly film and non-woven fabric, which are laminated together. The breathable poly film may comprise micro-porous film, 2-dimensional apertured film, 3-dimensional apertured film, hydro-entangled film, or any combination thereof. Such 3-dimensional apertured poly film are commercially available from Tredegar Film Products of Richmond, Va.

An acceptable sheet may also be a combination of layers of the materials listed above. Such combinations can be bonded in various ways, such as adhesive, co-hesive, ultrasonic bonding, chemical bonding, heat sealing, poly extrusion, etc.

The sheet of the present invention can be formed into various shapes, such as trapezoidal shapes wherein the third edge is wider in length than the fourth edge or vice versa. In addition, the sheets can be various lengths, but generally a length greater than its width. Preferably, the width of the sheet is at least about 4 inches and the length of the sheet is at least about 10 inches. Furthermore, the width of the sheet should be sufficient, when attached to the absorbent product, to mitigate slippage of the garment causing it to possibly leak.

The fastener used in the side panel of the present invention can be adapted to affix the side panel be an adhesive such as an adhesive fastening tape. For example, such adhesive fastening tapes are commercially available from 3M Personal Care (3M) of St. Paul Minn. The adhesive fastening tape may be combined with an adhesive release tape to create a Y-bond attachment configuration. The adhesive release tape is also commercially available from 3M.

In addition, the fastener can be adapted to affix to the side panel by way of mechanical hooks. One such mechanical hook is commercially available from 3M. Yet another commercially available product is Velcro®. The use of mechanical hooks requires a material to attach to such as a loop (brushed or knit) or some other type of engagable material such as a high loft non-woven. In another embodiment, the adhesive attachable tape and the mechanical attachment system can be combined. Both attachment means can be positioned along side one another allowing the user to use either or both. The attachment means can be positioned on either edge or both, and in the case of the one-piece side panel, can be positioned on both edges and in a middle portion.

In a further embodiment, the side panel of the present invention can additionally comprise an adjustable means. By adjustable means is meant, in the context of this invention, a mechanism that allows the side panel's length to be shortened. This can be accomplished by providing an adjustment fastener on the side of the panel opposite the fastener and then folding the side panel in a Z fold.

The absorbent article referred to herein can be a standard adult brief product such as DEPENDSυ, produced by Kimberly-Clark Corporation of Dallas, Tex. Other related incontinent products include Belted Undergarments and Refastenable Protective Underwear produced by Kimberly-Clark Corporation of Dallas, Tex. In addition, it may be possible to use the side panels of the present invention with lighter incontinent products such as Anatomical Pads (Pad & Pant Systems), produced by SCA Hygiene Products AB of Molndal, Sweden.

In another embodiment, the present invention can be a two-piece absorbent article. To save money, the absorbent piece can be substantially rectangular, with a front portion, a back portion and an absorbent piece. The basic construction of the absorbent piece can be similar to that of a standard adult diaper or a standard light incontinent product. These products generally comprise a liquid permeable top sheet, an absorbent core and a liquid impermeable back sheet. The construction can be similar to a standard adult diaper with leg cut outs, but leg cut outs would be an additional cost to consider. The product would have to be long enough to provide a front and back portion for the side panels to attach to and to be secured about the wearer as shown in FIG. 2.

Without further elaboration the foregoing will so fully illustrate my invention that others may, by applying current or future knowledge, adopt the same for use under various conditions of service.

1. An attachable panel for use with an absorbent article comprising:
a) a sheet of skin-friendly material, having:
i) a first edge and a second edge extending in a lengthwise direction;
ii) a third edge and a fourth edge extending in a widthwise direction;
iii) a middle portion positioned between the first, second, third and fourth edges; and
b) a fastener, positioned along the third edge, fourth edge and middle portion of the sheet, adapted to affix said sheet to an absorbent article positioned between the legs of a wearer, having a portion of the sheet positioned in the front and back of the wearer, wherein the back and front have a right and left edge,
wherein said panel attaches to the front or back portion and to the two edges of either the back or front portion of the article, depending on the orientation chosen, thereby securing the absorbent article about both sides of the wearer.

2. The attachable side panel of claim 1, wherein all or a portion of the sheet is elastic.

3. The attachable side panel of claim 1, wherein all or a portion of the sheet is a non-woven material.

4. The attachable side panel of claim 1, wherein the third edge and fourth edge have a width greater or narrower than the middle portion.

5. The attachable side panel of claim 1, wherein the sheet's length is greater than its width.

6. The attachable side panel of claim 1, wherein the sheet's length is at least about 10 inches.

7. The attachable side panel of claim 1, wherein the fastener affixes the sheet by an adhesive means or by a mechanical means or combination thereof.

8. The attachable side panel of claim 1, wherein the sheet's width is at least about 4 inches

9. The attachable side panel of claim 1, wherein the side panel additionally comprises an adjustable means.

10. A two-piece absorbent article, comprising:
a) a side panel comprising:
i) a sheet of skin-friendly material, having:
(a) a first edge and a second edge extending in a lengthwise direction;
(b) a third edge and a fourth edge extending in a widthwise direction;
(c) a middle portion positioned between the first, second, third and fourth edges; and
ii) a fastener adapted to affix said sheet along the third edge, fourth edge and middle portion to an absorbent article; and
b) an absorbent article having:
i) a center portion having an absorbent core positioned between the legs of a wearer;
ii) a front portion that is positioned in front of the wearer; and
iii) a back portion positioned in the back of the wearer,
wherein said side panel attaches to the front and back portion of said absorbent article securing it about both sides of the wearer.
